(19) United States
(12) Patent Application Publication
(10) Pub. No.: US 2026/0259207 A1
(43) Pub. Date: Sep. 3, 2026

(54) DEVICE AND METHOD FOR DETECTING ADULTERATION URINE BY IMMUNOASSAY

(71) Applicants: TOPAZ DIAGNOSTICS, INC., CLAYMONT, DE (US); Hangzhou Biotest Biotech Co., LTD., Hangzhou (CN)

(72) Inventors: Matthias VOLK, Halibut Bay (NS); Junsheng WU, Hangzhou (CN)

(21) Appl. No.: 19/655,972

(22) Filed: Apr. 23, 2026

Related U.S. Application Data
(63) Continuation-in-part of application No. 18/920,432, filed on Oct. 18, 2024.

(30) Foreign Application Priority Data
Oct. 20, 2023 (CN) 202311368227.7

(51) Int. Cl. G01N 33/543; G01N 33/53; G01N 33/58; G01N 33/94

(52) U.S. Cl. CPC G01N 33/54388; G01N 33/5308; G01N 33/582; G01N 33/583; G01N 33/9413; G01N 2470/04; G01N 2470/10

(57) ABSTRACT

This invention provides a device for detecting whether urine has been adulterated, said device comprising means for detecting the presence or quantity of an adulteration marker in the urine, wherein said device tests for the presence or quantity of the adulteration marker based on an immune reaction.

### CROSS-REFERENCE TO RELATED APPLICATIONS

The patent application is the continue in part of the US application with the Ser. No. 18/920,432, filed on Oct. 18, 2024, which claims a prior application in China, Application No.: 202311368227.7, filed on Oct. 20, 2023, the specification, abstract, claims, and accompanying drawings of which are incorporated by reference in their entirety as a part of this application.

### BACKGROUND OF THE INVENTION

### Field of the Invention

The present invention relates to the field of in vitro diagnosis, and in particular to a device and method for testing whether a urine is adulterated by using immunological methods.

### Description of the Related Art

The following introduction to the background art is merely an introduction to some common background knowledge and does not constitute any limitation to the present invention.

Currently, detection devices or methods for detecting whether a sample contains an analyte are widely used in hospitals or homes. These detection devices for rapid diagnosis include one or more test strips, such as those for early pregnancy testing, drug abuse testing, and the like. Such rapid diagnostic detection devices are very convenient, and test results can be obtained on the test strips in about one minute, or at most ten minutes. At present, with the popularity of home testing, many such rapid diagnoses can be performed at home.

In the field of drug abuse, lateral flow and immunological methods are used to test for some analytes related to drug abuse. Generally, during urine testing, the test subject usually does not want a positive result, and water or other substances are added to the collected urine sample to affect the normal testing of small drug molecules. To solve this problem, traditional technologies generally require additional testing for urine adulteration, which usually adopts chemical reactions to test whether the urine is adulterated.

For example, a U.S. published invention patent, Publication No.: US2015/0140681, discloses the use of a color reaction to test the pH value of urine. If the pH value is not within the normal range, it is considered to be suspected of adulteration. The method for testing pH also only adopts chemical methods, such as chemical reactions to generate colored substances to indicate the pH value. For example, a U.S. published invention patent, Publication No.: US2004/0018636A1, discloses a test strip different from immunological tests for testing urine adulteration, and the reaction principle for testing urine adulteration also adopts chemical methods.

When performing immunological testing to detect analytes in urine sample, chemical reactions are used to test if the urine sample is adulterated or not, and two different reaction systems test the same urine sample. This has the following disadvantages: First, both methods need to contact the same urine sample. Sometimes, chemical reaction testing for adulteration needs to be immersed in urine, and some chemical substances will dissolve in the urine sample. These chemical substances will interfere with the performance of the immune reaction and directly affect the accuracy of the immunological reaction in testing the analyte in the urine sample. In addition, in terms of production, immunological testing for analytes requires one production line, while chemical reaction-based adulteration testing requires another independent production line. Due to different raw material and environmental requirements, independent production lines are needed, which significantly increases the production cost. In addition, in the design of the testing device, it is necessary to separately set up separate locations for the test strips for immunological testing of analytes and those for adulteration testing, which may inevitably increase the design and production costs of the manufacturing device.

Therefore, it is necessary to provide a simpler device that can simultaneously test for sample adulteration, especially urine adulteration, when using immunological methods to test for analytes in urine sample, making the test more accurate.

### BRIEF SUMMARY OF THE INVENTION

To overcome some technical defects of traditional technologies, the present invention provides a device for detecting whether urine sample (the urine is the natural excretion of an individual or synthetic urine/artificial urine) is adulterated. The device includes detecting the presence or quantity of a urine adulteration marker, and the device tests the presence or quantity of an adulteration marker based on immune reactions. In some embodiments, the device further includes testing the presence or quantity of an analyte in the urine sample, and the testing of the analytes is also performed using immunological methods.

In some embodiments, the immunological testing method is implemented based on the principle or mechanism of mutual recognition or binding between antibodies or antigens.

In some embodiments, the device includes a lateral flow test strip, and based on the lateral flow test strip, the presence or quantity of adulteration markers in urine is tested using immunological methods.

In some embodiments, the lateral flow test strip includes antibodies and/or antigens, or first or second receptors that bind to an adulteration marker, and the binding between the receptor and the adulteration marker is used to test the presence and quantity of the adulteration marker.

In some embodiments, the immunological testing method includes a competitive binding mode or a non-competitive binding mode.

In some embodiments, the test strip includes a labeling region and a test region. The test region has a second receptor immobilized thereon; the labeling region includes a first receptor that can specifically bind to the adulteration marker in urine and form a complex with the adulteration marker. In some embodiments, the complex and the first receptor can flow with the liquid sample (urine sample), while the second receptor cannot flow with the liquid sample. When the adulteration marker is present in the urine, the complex is captured on the test region to form a positive result, indicating that the urine is not adulterated; if the test region shows a negative result, it indicates that the urine is adulterated.

In some embodiments, the adulteration marker or its analog is immobilized on the test region. When the adulteration marker is not present in the urine or is less than a preset threshold, the adulteration marker or its analog on the test region captures the first receptor to form a negative result, indicating that the urine is adulterated; when the adulteration marker is present in the urine or is greater than the preset threshold, the adulteration marker in the urine competes with the adulteration marker or its analog immobilized on the test region to bind to the first receptor, forming a positive result on the test region, indicating that the urine is not adulterated.

In some embodiments, the first receptor is labeled with a labeling substance, and the presence or quantity of the adulteration marker in urine is indirectly measured by measuring the labeling substance. In some embodiments, the labeling substances include dyes (water-soluble dyes), fluorescent dyes, and colored particle labeling substances. The colored particle labeling substances are latex particles or metal particles, and in some embodiments, the metal particles include gold particles. In some embodiments, the first receptor is conjugated to the labeling substance.

In some embodiments, the adulteration markers are endogenous metabolites (produced by the body itself) are present in a biological urine sample, such as urine. In some embodiments, the metabolites include one or more inorganic small molecules and organic substances. In some embodiments, the metabolites have a normal concentration range in urine. In some embodiments, the biological individual includes healthy or unhealthy populations. In some embodiments, the so-called normal concentration range of surrogate substances in urine includes the normal concentration range of healthy people or the normal concentration range of unhealthy people.

In some embodiments, if the quantity or concentration of the adulteration marker tested is not within the normal range, it may indicate that the urine is adulterated. In some embodiments, if the adulteration marker is less than the normal range, it indicates that the urine is adulterated; alternatively, in some embodiments, if the adulteration marker is greater than the normal concentration range, it indicates that the urine is adulterated. In some embodiments, when the concentration of the adulteration marker in urine decreases and is not within the normal range, it indicates that the urine may be adulterated.

In some embodiments, an “adulteration concentration range” for the adulteration marker is set compared with the “normal concentration range” of the adulteration marker. If the tested adulteration marker is within the adulteration concentration range, it indicates that the urine may be adulterated; if it is within the normal concentration range, it indicates that the urine is not adulterated.

In some embodiments, the urine includes the urine naturally excreted from the individual (called as natural urine) and a synthetic/artificial urine (no natural urine).

In some embodiments, the endogenous metabolites present in nature urine include nitrogen-containing metabolites, carbohydrates, lipid metabolites, electrolytes and inorganic ions, and microorganisms.

In some embodiments, the nitrogen-containing metabolites include urea, creatinine, uric acid, ammonia, amino acids, and peptides. In some embodiments, the uric acid metabolites include purine substances, which include adenine, guanine, hypoxanthine, xanthine, adenosine, uridine, and other metabolites. In some embodiments, the metabolites include pterin metabolites, which include neopterin, biopterin, dihydroneopterin, dihydrobiopterin, sepiapterin, or 7-hydroxypterin.

In some embodiments, the glucose metabolism markers include glucose, ketone bodies, and lactic acid; the oxidative stress/immune markers include neopterin (NPT), malondialdehyde (MDA), and 8-OHdG; the enterohepatic circulation markers include urobilinogen and bilirubin; the hormone and endocrine markers include catecholamines, steroid metabolites, or dopamine.

In some embodiments, the test region includes a nitrocellulose membrane, and a test result control region is included downstream of the test region. In some embodiments, the presence and quantity of the adulteration marker are determined by the shade or depth of the color on the test region. In some embodiments, whether the urine is adulterated is judged by comparing the colors of the test region and the test result control region.

On the other hand, the present invention provides a method for testing whether a urine is adulterated. The method includes: testing an adulteration marker in urine by immunological methods. When the adulteration marker is within the normal range, it indicates that the urine is not adulterated; when the adulteration marker is not within the normal range, it indicates that the urine is adulterated.

In some embodiments, a first receptor and a second receptor are provided, wherein the first receptor specifically binds to the adulteration marker in urine to form a complex, and the second receptor specifically binds to the complex formed by the first receptor and the adulteration marker; alternatively, the complex formed by the first receptor and the adulteration marker competes with the adulteration marker to bind to the second receptor.

In some embodiments, the immunological testing method is completed on a lateral flow test strip. In some embodiments, the lateral flow test strip includes a labeling region and a test region located downstream of the labeling region. The test region has a second receptor immobilized thereon, which cannot flow with the liquid, while the labeling substance is conjugated to the first receptor, which can flow with the liquid.

In some embodiments, the first receptor may include an antibody or an antibody analog, and the second receptor may be an antibody, an antigen, or an analog of the adulteration marker.

When the urine sample is synthetic urine, an immunological test for an adulteration marker that yields a negative result indicates the synthetic urine is not adulterated, whereas a positive result indicates the synthetic urine has been adulterated.

When the urine sample is natural urine, an immunological test for an adulteration marker that yields a negative result indicates the natural urine is adulterated, whereas a positive result indicates the synthetic urine is not adulterated.

### Beneficial Effects

The present invention uses immunological methods to test for adulteration markers in urine, which can be combined with the normal testing of analytes in urine to form a testing device. This avoids the risk of cross-contamination when two different testing systems (chemical and immunological) are used for testing. In addition, the same testing technology principle is used, only the purpose of detection is different, which reduces production costs and reduces cross-contamination. Finally, using the present invention to test if a nature urine or synthetic urine is adulterated by adding adulteration markers into the synthetic urine or diluting the natural urine as to reduce the concentration of the adulteration markers in the natural urine.

### BRIEF DESCRIPTION OF THE DRAWINGS

FIG. 1 is a schematic structural diagram of a lateral flow test in a specific embodiment of the present invention.

FIG. 2 is a schematic structural diagram of a lateral flow test in a specific embodiment of the present invention.

FIG. 3 is a schematic structural diagram of a lateral flow test in a specific embodiment of the present invention.

FIG. 4 is a diagram showing the test result of naturally excreted urine from a human being using the lateral flow test of the present invention.

FIG. 5 is a schematic diagram of commercial artificial urine. It can itself be used to adulterate and dilute naturally produced urine; it can also be adulterated itself to be used as naturally produced urine (for example, by adding the adulteration markers of the present invention to counterfeit natural urine).

FIG. 6 is a schematic diagram of a negative result by testing a traditional commercial artificial urine using the test strip of the present invention.

FIG. 7 is a comparison diagram of the test results of naturally excreted human urine and currently commercial artificial urine using the test strip of the present invention (the left side commercial artificial urine, all tests show negative results, indicating that it is not naturally excreted urine but artificial urine; among them, test strip 2063 uses NPT as the adulteration marker, and test strip 2064 uses another adulteration marker (dopamine resp. Homovanillic Acid; HVA); the right side is normal urine, using NPT as the adulteration marker (test strip 2062), a positive result is obtained; using dopamine/HVA to test normal urine, no line appears, indicating a positive result, indicating that the normal natural urine is not adulterated).

FIG. 8 is a color chart for reading the depth of the color line on the test region.

### DETAILED DESCRIPTION OF THE INVENTION

The structures involved in the present invention or the technical terms used are further explained below. Unless otherwise specified, they shall be understood and explained according to the general terms commonly used in the art.

### Detection

Detection refers to assaying or testing for the presence of a substance or material, such as, but not limited to, chemical substances, organic compounds, inorganic compounds, metabolites, drugs or drug metabolites, organic tissues or metabolites of organic tissues, nucleic acids, proteins, or polymers. In addition, detection refers to testing the quantity of a substance or material. Furthermore, assaying also refers to immunological detection, chemical detection, enzyme detection, and the like.

### Detection Method

Any known method can be used to test the adulteration markers of the present invention, which are present in liquid samples, such as urine sample. The testing method can use, for example, isotope methods: the method established by Resume et al. in 1985, or chromatographic methods: high-performance liquid chromatography (HPLC) is a relatively mature and widely used method. The disadvantage is that there are many variations in sample processing, chromatographic conditions, sample detection, and quantification, making it difficult to standardize. Fully automatic high-performance liquid chromatography is also a method. HPLC can be divided into various methods according to the derivation method (pre-column or post-column derivation) and detection method (fluorescence, electrochemistry). HPLC is used to accurately determine the adulteration markers of the present invention. The testing methods also include immunological methods: this method applies specific anti-adulteration marker technologies, such as monoclonal antibodies, and uses fluorescence polarization or immunological methods to determine the adulteration markers.

In some embodiments, immunological methods are used to test for an adulteration marker. The so-called “immunological” method refers to a method that uses the specific binding reaction between antigens and antibodies, or a method similar to the binding of antigens and antibodies. Such tests rely only on physical interactions or biological specific binding, and the structure of the two substances does not change or does not undergo substantial changes before and after binding.

Traditional chemical testing methods refer to testing methods that achieve qualitative, quantitative, or characteristic analysis of the target substance based on chemical changes by causing chemical reactions, molecular structure changes, chemical bond breakage or formation of the tested substance, forming new chemical substances or generating detectable changes in chemical signals, such as color development tests, or testing by generating new colored substances through redox reactions.

In some embodiments, the present invention uses immunological methods to test for an adulteration marker in urine, rather than traditional chemical methods. This is a testing platform that can be uniformly used with the existing detection of analytes in urine, only with different purposes, which is also the core of the present invention. In this way, in one testing system, immunological methods can be used to test for analytes in urine sample and simultaneously test for an adulteration marker as to determine if the urine is adulterated or not. In some embodiments, the analytes tested in urine by immunological methods are exogenous substances that enter the human body and are metabolized into urine, such as some drug abuse substances. The adulteration marker in urine are substances produced by the metabolism of the biological individual itself for testing.

The immunological methods include fully automatic chemiluminescent immunoassay technology, which uses instruments to detect adulteration markers. This method is fast, simple to operate, and highly automated, which can reduce human error, has good accuracy and precision, and is suitable for most clinical laboratory applications.

In some embodiments, chromatographic technology can be used, which is a disposable test strip that uses dry substances processed on a lateral flow test strip to perform chromatography when encountering water, also known as a lateral flow test strip. The testing of a urine adulteration substance is performed through the flow of liquid on the strip. Generally, test result can be obtained quickly within 3-10 minutes using this test strip, and it is more suitable for non-professional testing, such as home self-testing, or OTC products, with fast results and convenient operation.

### Sample

the detection method or detection device of the present invention can detect one or more combinations of adulteration markers in biological samples. The biological samples here include biological fluids (such as clinical fluids or clinical samples). Liquid samples or fluid samples can be derived from solid or semi-solid samples, including excreta, biological tissues, and food samples. Any appropriate method can be used to convert solid or semi-solid samples into liquid samples, such as mixing, mashing, macerating, incubating, dissolving, or digesting solid samples with enzymatic hydrolysis in a suitable solution (such as water, phosphate solution, or other buffer solutions). “Biological samples” include those derived from animals, plants, and food samples, such as urine, saliva, blood and its components, cerebrospinal fluid, vaginal secretions, sperm, feces, sweat, secretions, tissues, organs, tumors, cultures of tissues and organs, cell cultures, and media. Preferably, the biological sample is urine, and more preferably, the biological sample is saliva. Food samples include food processing substances, final products, meat, cheese, wine, milk, and drinking water. Plant samples include those derived from any plants, plant tissues, plant cell cultures, and media. “Environmental samples” are derived from the environment (for example, liquid samples from lakes or other water bodies, sewage samples, soil samples, groundwater, seawater, and waste liquid samples). Environmental samples may also include sewage or another wastewater.

Any analyte can be detected using a suitable detection element or test element of the present invention. Preferably, the present invention is used to detect analytes in saliva and urine. In some embodiments, immunological methods are used to test for adulteration markers in urine to test whether the urine is adulterated, thereby assisting the testing of analytes in urine.

### Downstream and Upstream

Downstream or upstream is divided according to the direction of liquid flow; generally, liquid flows from the upstream to the downstream region. The region located downstream receives liquid from the upstream region, and liquid can also flow from the upstream region to the downstream region. Here, it is generally divided according to the direction of liquid flow. For example, on some materials that promote liquid flow by capillary force, liquid can flow in the direction opposite to gravity due to gravity. At this time, upstream and downstream are still divided according to the direction of liquid flow. For example, in the detection device of the present invention, when the absorbent element absorbs the fluid sample, the fluid 100 can flow from the liquid outlet of the capillary channel into the detection chamber, contact the test element 300 above, and flow to the sample application region 205 of the test element 300. At this time, the flow of liquid from the sample application region 205 to the absorption region 201 is from upstream to downstream. During the flow, it passes through the labeling region 204 and then flows to the test region 202, where the test region has a detection region 206 and a test result control region 207. The test region 202 may be a polyester fiber membrane, and the sample application region 205 may be glass fiber.

### Gas Communication or Liquid Communication

Gas communication or liquid communication means that liquid or gas can flow from one place to another, and during the flow, some physical structures may act as guides. The so-called passing through physical structures generally means that liquid passes through the surface of these physical structures or the internal space of these structures to flow passively or actively to another place. Passive flow is generally caused by external forces, such as flow under capillary action. The flow here can also be due to the liquid or gas's own action (gravity or pressure), or passive flow. The communication here does not mean that liquid or gas must exist, but only indicates the connection relationship or state between two objects in some cases. If liquid exists, it can flow from one object to another. Here, it refers to the state of connection between two objects. On the contrary, if there is no liquid communication or gas communication between two objects, if there is liquid in or on one object, the liquid cannot flow into or onto the other object, and this state is non-communication, non-liquid or non-gas communication.

### Urine Adulteration Marker

The urine adulteration markers of the present invention refer to some substances present in urine naturally excreted by the human body (called as natural urine), which have a normal concentration range. When natural urine is adulterated, such as adding water or other traditional artificial urine that is not natural urine to dilute the natural urine, thereby reducing its concentration to be outside the normal concentration range, the value tested by the reagent is outside the normal concentration range, indicating that the urine is adulterated. If the urine is adulterated, it is equivalent to reducing the concentration of the analyte or the concentration of the adulteration marker, and the tested analyte has a lower concentration than that of unadulterated urine, or even cannot be tested. For example, replacing urine naturally excreted by the human body with some liquids that do not contain adulteration markers (such as commercial artificial urine) to make the adulteration markers absent, thereby avoiding the suspicion of adulteration.

Therefore, the urine adulteration markers of the present invention are an auxiliary test to keep the test result of the analyte consistent with the true result, or to assist in explaining whether the test result of the analyte is correct. For example, when a drug user undergoes urine testing, he always hopes not to be detected as a positive result, so he adds liquids that do not contain drugs, such as tap water and artificial urine, to his own excreted urine to dilute the urine, so that the concentration of drugs is reduced to an undetectable level.

In some embodiments, the urine adulteration markers of the present invention are substances produced by the metabolism of the biological individual itself (endogenous), which are naturally excreted through the urethra, rather than substances metabolized endogenously in the biological individual through the intake of exogenous substances (such as oral or injected drugs and their metabolites, food additives, preservatives, environmental pollutants, caffeine, alcohol, tea polyphenols and other exogenous substances).

The so-called urine adulteration here mainly refers to diluting naturally excreted urine with using liquids that do not contain exogenously ingested drugs or their metabolites (such as artificial urine) as to reduce the concentration of exogenous drugs. For example, adding water to urine, including tap water, mineral water, purified water, or drinking a large amount of water in a short time, or adding white vinegar, lemon juice, citric acid, hydrogen peroxide, disinfectant, soapy water, detergent and other liquids to dilute the natural urine, or replacing naturally excreted urine with commercial artificial urine.

When chemical testing is used, adding bleach, disinfectant, hydrogen peroxide, etc., will oxidize the analyte, thereby reducing the analyte. Adding white vinegar, lemon juice, citric acid will interfere with some redox enzyme reactions, leading to the failure of chemical methods to test for adulteration. However, using immunological methods to test for endogenous adulteration markers, whether the urine is diluted or exogenous oxidants are added, the abnormal changes in the concentration of adulteration markers can be detected, and the urine adulteration can be tested more accurately.

The present invention is primarily intended for testing adulteration of urine naturally produced by individuals, and may of course also be used for testing adulteration of artificial urine. Another type of adulteration behavior is purchasing commercial artificial urine. Such urine is not naturally excreted from the human body, but is artificially prepared to be similar to urine excreted by the human body, containing various indicators of human urine, such as specific gravity and pH value, but lacking some endogenous metabolites of the human body that are present in naturally excreted human urine but not in commercial artificial urine. Thus, when using chemical methods to test for urine adulteration, such as testing specific gravity and pH value, the test results are the same as those of naturally excreted human urine, resulting in the inability of chemical methods to effectively identify whether the urine is adulterated. When using immunological methods to test for endogenous adulteration markers, these markers are not present in artificial urine, and a negative result appears, which can effectively test for urine adulteration.

In some embodiments, since they are endogenous metabolites of biological individuals and are naturally excreted in urine, when the biological individual (mammal or human) is healthy, these substances have a healthy “normal concentration range”, which generally does not fluctuate greatly. Of course, if the biological individual is unhealthy or a certain disease occurs, some endogenous metabolites will increase or decrease, and these increases and decreases always belong to a “normal concentration” range under unhealthy or disease conditions, which can be detected.

Therefore, in some embodiments, the so-called “normal concentration range” of adulteration markers in the present invention includes these endogenous adulteration markers present in naturally excreted urine. In addition, whether the individual is healthy, unhealthy, has a disease, or already has a disease, the adulteration markers in the urine are always within a range, which is the so-called “normal concentration range” of the present invention. Therefore, the so-called “normal concentration range” of adulteration markers here is opposite to the so-called “adulteration concentration range” of adulteration markers in urine adulteration. Generally, the “adulteration concentration range” is not within the normal concentration range but outside the normal range, such as less than the normal range.

In some embodiments, if the tested adulteration marker is within the adulteration concentration range, it indicates that the urine is adulterated; if it is within the normal concentration range, it indicates that there is no adulteration. The adulteration range can be 0 or less than the normal concentration range. The unit of the range here can be the concentration range of the adulteration marker, such as grams, milligrams, micrograms per liter, per milliliter, etc.

In some embodiments, the urine of the present invention also includes commercial artificial urine, and the adulteration behavior may also include adulteration of artificial urine. Through research by the inventors of the present invention, conventional commercial artificial urine currently does not contain the endogenous adulteration markers of the present invention. However, if such artificial urine contains the endogenous markers of the present invention, it indicates that the artificial urine has been adulterated. Generally, the absence of endogenous markers of the human body in artificial urine indicates that the content of adulteration markers in the artificial urine is within a “normal concentration range”, for example, the content of adulteration markers is zero or below a predetermined threshold. The presence of the endogenous adulteration markers of the present invention in artificial urine indicates that the content of adulteration markers in the artificial urine is within an “adulterated concentration range”, for example, the content is greater than zero or above a predetermined threshold. For example, when the immunological test strip of the present invention is used to test artificial urine, a positive test result indicates that the artificial urine has been adulterated, and a negative test result indicates that the artificial urine has not been adulterated. This is opposite to the result for naturally excreted urine: a positive result for naturally excreted urine indicates that the urine has not been adulterated, while a negative result indicates that the urine has been adulterated.

Therefore, “urine” in the present invention includes both naturally excreted urine from an individual and commercial artificial urine. The object of adulteration may be naturally excreted urine or artificial urine. Different objects of adulteration lead to different immunological test results and thus different judgments of adulteration. Thus, if conventional commercial artificial urine is found to contain the adulteration markers of the present invention, it indicates that the artificial urine has been adulterated, which also falls within the scope protected by the claims of the present invention.

In the present invention, unless otherwise specified, urine adulteration refers to adulteration of naturally excreted urine from an individual; and where expressly specified, it also includes adulteration of artificial urine.

In some embodiments, for the endogenous metabolites, when a disease occurs, their concentration is generally higher than that in healthy individuals, and they can also be used as natural urine adulteration markers of the present invention. For example, an adulteration marker named A has a concentration range of 50-100 mg/mL in naturally excreted urine under healthy conditions (healthy normal range). When the individual has a disease, it increases to more than 150 mg/mL or higher than 100 mg/mL (unhealthy normal range). The healthy normal range and the unhealthy normal range are collectively referred to as the “normal concentration range” of the adulteration marker. In this embodiment, the normal range is 50-100 ng/mL and concentrations higher than 100 ng/mL, such as 150, 180, 200, which are all the “normal range” of the present invention. The “adulteration range” of urine is the range less than 50 ng/mL, which can be considered as the adulteration range. This is an example to illustrate or explain the definition of the present invention, and the normal range of the present invention is different from the urine adulteration concentration range. For example, adding water to naturally excreted urine to dilute the urine, naturally reducing the normal range of the adulteration marker from the normal range to the adulteration range, thereby testing whether the adulteration marker exists and whether it is within the adulteration range. If it is within the adulteration range, it indicates that the urine is adulterated; if it is not within the adulteration range, it indicates that there is no adulteration.

If tested by the immunological method of the present invention, the detected concentration of A is less than 50 ng/mL, or the test result is negative. Here, the test threshold can be set to 50 ng/mL or less than 50 ng/mL, such as 45 ng/mL or 30 ng/mL, or 0. When the detected value of the adulteration marker is higher than the set threshold, it is a positive result, indicating no adulteration; when the test result is negative, it indicates adulteration. It can be understood that the positive and negative results are related to whether a line appears in the test region. When the competitive method is used, the appearance of a line in the test region indicates a negative result, and the absence of a line indicates a positive result or the color of the line is very light. When other non-competitive methods are used, the appearance of a line indicates a positive result, and the absence of a line indicates a negative result. If a positive result cannot be detected but a negative result is obtained, this is obviously an abnormal decrease in concentration, and it is very likely that the urine has been artificially added with water or other non-urine to dilute the urine, such as adding tap water to reduce its concentration, thereby reducing the concentration of the adulteration marker to outside the normal range or less than the normal range, which can indicate that the natural urine is adulterated. Of course, if the detection result is higher than 100 ng/mL or a positive result, it indicates no adulteration, but it may indicate the occurrence of a certain disease.

Therefore, in some embodiments, some marker substances present in urine for disease diagnosis can also be used as marker substances for urine adulteration (endogenous metabolites). For example, when a disease occurs or is present, the concentration of disease diagnosis markers in urine will increase compared with that in healthy individuals. If the actual detection finds that it is lower than the normal range of healthy individuals, it can indicate urine adulteration. Although some endogenous markers in urine are associated with diseases and can be used for disease diagnosis, they can also be used for natural urine adulteration, which is first discovered and used in the present invention.

Some adulteration markers cannot be tested by chemical reactions, but using immunological methods is easier and more feasible. This reflects that the present invention uses immunological methods for adulteration testing, which relatively broadens the methods for urine adulteration testing. After all, immunological testing is based on the principle of antigen-antibody binding and does not produce new substances like chemical methods. The testing of exogenous substances metabolized in the individual is generally the testing of analytes. Some endogenous metabolites and exogenous metabolites are tested by immunological methods, which obviously reduces the interference of test results and avoids mutual interference of test results (traditionally, chemical methods and immunological methods are used in one test system at the same time).

Of course, there are other substances in urine that, whether healthy or diseased, always have a stable concentration range or little change. Such substances can also be used as urine adulteration markers. Their concentration in urine is in a stable range. When tested, if it is less than the stable range, it indicates that the urine may be adulterated.

In some embodiments, urine generally contains 95% water, and the remaining 5% is solids. Most of these solids are endogenous metabolites, and there are also metabolites of exogenous drugs (these are generally analytes). The urine adulteration markers of the present invention generally use endogenous metabolites, rather than exogenous drugs and their metabolites as adulteration markers.

In some embodiments, the endogenous metabolites in urine that can be used as adulteration markers include nitrogen-containing metabolites, such as urea (15-40 mg/mL); intermediate or end products of purine metabolism that cause uric acid; amino acids and their derivatives, such as amino acids (glycine, alanine), amino acid metabolites, such as homocysteine (100~1500 ng/mL), taurine (10~100 μg/mL); purine/pyrimidine metabolites, such as adenine, guanine, hypoxanthine (0.5~3 μg/mL), xanthine (1~5 μg/mL), neopterin (NTP) (0.01~0.05 μg/mL), biopterin (0.005~0.05 μg/mL). Most of the above metabolites are small chemical molecules, which cannot be tested by chemical reactions or generally by redox methods, but can be tested by immunological methods of the present invention.

Endogenous metabolites can also be peptides and proteins, such as trace small molecular peptides and urinary proteins. Such substances are also endogenous metabolites and are in a normal range in naturally excreted urine. They can be tested by the double antibody sandwich method. When the test result is positive, it indicates that the urine is not adulterated; if the test value is negative, it indicates that the urine is adulterated.

In some embodiments, the substances in urine that can be used as adulteration markers include endogenous carbohydrates and their metabolites, including glucose (normal trace amount, glycosuria indicates abnormality, <20 μg/mL), lactose, fructose, galactose and other sugars, glycolysis/gluconeogenesis products such as lactic acid, pyruvic acid, citric acid; ketone bodies such as acetoacetic acid, β-hydroxybutyric acid, acetone; glycosamines/glycoconjugates such as glucuronic acid. Some of the above substances are also small molecular compounds and cannot be tested by chemical reactions.

In some embodiments, the substances in urine that can be used as adulteration markers include endogenous lipids and their metabolites, including: short-chain/medium-chain/long-chain fatty acids, keto acids and other fatty acids; glycerophospholipid metabolites such as glycerol, glycerophosphate, choline; steroids and bile acid metabolites such as cholesterol, bile acids (enterohepatic circulation markers), sex hormone/cortisol metabolites.

In some embodiments, the substances in urine that can be used as adulteration markers include electrolytes and inorganic metabolites of endogenous substances, such as cations such as Na⁺ (30~200 μg/mL), K⁺ (20~80 μg/mL), Ca²⁺ (10~30 μg/mL), Mg²⁺ (5~15 μg/mL), NH₄⁺, anions such as Cl⁻ (50~250 μg/mL), HCO₃⁻, phosphate (PO₄³⁻), sulfate (SO₄²⁻); trace elements such as iron, zinc, copper, selenium.

In some embodiments, the substances in urine that can be used as adulteration markers include vitamins and coenzyme metabolites of endogenous substances, including water-soluble microorganisms such as metabolites of B vitamins (B1/B2/B6/B12), vitamin C, folic acid; fat-soluble vitamins such as metabolites of vitamins A/D/E/K; auxiliary derivatives such as metabolites of NAD⁺ and FAD. These substances can also be tested by immunological methods, but currently, they are relatively difficult to test by chemical reactions.

In some embodiments, the substances in urine that can be used as adulteration markers include hormones and signal molecule metabolites of endogenous substances, including steroid hormones, such as metabolites of estradiol, testosterone, cortisol (such as 17-hydroxycorticosteroids 0.01~0.05 μg/mL); peptide hormones, such as epinephrine, norepinephrine, dopamine (DOP) (32.5~400 ng/mL) and their metabolites (such as vanillylmandelic acid VMA (0.5~2 μg/mL)); thyroid hormones, such as metabolites of T3 and T4. Most of the above endogenous metabolites are small chemical molecules. These substances are generally not present in commercial artificial urine, and their range in naturally excreted human urine is relatively stable. Therefore, using these small chemical molecule adulteration markers can also be used for urine adulteration testing, especially for testing the replacement of naturally excreted individual urine with commercial artificial urine.

In some embodiments, the normal ranges of the above substances used as urine adulteration markers can generally be tested in advance according to populations and ages in different regions, or determined according to the concentration ranges of some disease diagnosis markers. In this way, the normal range can be easily obtained. With the normal range, it is naturally easy to set the interval of the “adulteration range”, so that these markers can be used as adulteration markers, and the testing method adopts immunological testing of adulteration markers.

### Analyte

The analytes here include exogenous metabolites in urine, including food additives, plant components, spices or condiments, dairy products, drugs and their metabolites, drugs and metabolites of abuse substances.

In some embodiments, food additives include benzoic acid, sorbic acid, saccharin, aspartame metabolites, tartrazine, sunset yellow; plant components include caffeine, theobromine, theophylline, polyphenols, flavonoids, coumarins, phytosterols; spices or condiments include menthol, cinnamic acid, anisic acid, allicin metabolites; drugs for medical treatment of diseases and their metabolites include antipyretic and analgesic drugs, antibiotics, psychotropic drugs, cardiovascular drugs, traditional Chinese medicine/natural medicine drugs.

In some embodiments, immunological methods can be used to test for drugs and drug abuse. The drugs or drug abuse here are ingested exogenously (orally or by injection) into the human body and metabolized. The “drug of abuse” (DOA) in the present invention refers to the non-medical use of drugs (usually having a nerve-paralyzing effect). Abuse of these drugs can cause physical and mental damage, dependence, addiction, and/or death. Examples of drug abuse include cocaine; amphetamines (AMP) (e.g., Black Beauties, white amphetamine tablets, dextroamphetamine, dextroamphetamine tablets, Beans); methamphetamine (MET) (crank, methamphetamines, crystal, speed); barbiturates (BAR) (e.g., Valium®, Roche Pharmaceuticals, Nutley, New Jersey); sedatives (i.e., sleep aids); lysergic acid diethylamide (LSD); depressants (downers, goofballs, barbs, blue devils, yellow jackets, methaqualone); tricyclic antidepressants (TCA, i.e., imipramine, amitriptyline, and doxepin); 3,4-methylenedioxymethamphetamine (MDMA); phencyclidine (PCP); tetrahydrocannabinol (THC, pot, dope, hash, weed, etc.); opiates (i.e., morphine (MOP), opium, cocaine (COC), heroin, oxycodone); anxiolytics and sedative-hypnotics. Anxiolytics are a class of drugs mainly used to reduce anxiety, tension, fear, stabilize mood, and have hypnotic and sedative effects, including benzodiazepines (BZO), atypical BZ, fused diazepine NB23C, benzazepines, BZ receptor ligands, open-loop BZ, diphenylmethane derivatives, piperazine carboxylates, piperidine carboxylates, quinazolinones, thiazine and thiazole derivatives, other heterocycles, imidazole-type sedatives/analgesics (such as oxycodone (OXY), methadone (MTD)), propylene glycol derivatives-carbamates, aliphatic compounds, anthracene derivatives, etc. The detection device of the present invention can also be used to detect substances that are for medical use but are prone to overdose, such as tricyclic antidepressants (imipramine or analogs) and acetaminophen.

After these drugs are absorbed by the human body, they will decompose into different small molecular substances, which are present in body fluids such as blood, urine, saliva, sweat, or some body fluids. The above analytes are all exogenously ingested substances by the human body, which will be metabolized into small drug molecules in the human body, but are not endogenous substances.

### Urine Adulterating and Analytes in Urine

In some embodiments, the urine adulteration markers of the present invention are not the same as the analytes in urine. The adulteration markers are used to test whether the urine is adulterated, especially for testing the adulteration behavior of diluting natural urine by adding water or replacing naturally excreted urine of natural persons with commercial artificial urine. Of course, it can also test substances not contained in artificial urine. The analytes in urine are generally used to test for the presence and quantity, to indicate whether an individual has a certain health condition, such as whether they have a disease, or other conditions, such as drug abuse. In addition, the adulteration markers are endogenous, while the analytes are exogenous. The content of exogenous analytes in urine is directly affected by the amount ingested (or their metabolites), while endogenous substances are generally naturally metabolized in the human body and are generally not affected by the intake of exogenous substances. Their normal range is relatively constant, and at most, when the human body is healthy or unhealthy, there is a fluctuation, but the fluctuation range is also relatively fixed, with a fixed range.

In some embodiments, the analytes specifically refer to drug abuse substances or small molecules in urine. Generally, the purpose of testing for drug abuse is to detect whether the subject has drug abuse behavior, which is mainly in the scope of drug control, drug trafficking, etc. There are also some cases where job applicants are required to undergo urine drug testing during recruitment. These subjects do not want to be detected as drug users, and often add tap water to the collected naturally excreted urine to dilute and reduce the concentration of drug abuse metabolites. For example, 50 milliliters of collected urine can be poured out 30 milliliters, then 30 milliliters of tap water or artificial urine is added. It looks like natural urine from the outside, but in fact, adulteration has been carried out, so that the analyte cannot be detected or a negative result is obtained.

Thus, generally, in the traditional prior art, urine adulteration is tested by chemical methods, such as redox methods to test some adulteration markers in urine. Such adulteration markers are mainly oxidants, but these oxidants are exogenously added to naturally excreted urine and are not endogenous markers as this invention (for example, exogenously added bleach, hydrogen peroxide, hexavalent chromium, sodium hypochlorite, etc., will destroy drug metabolites and antigen/antibody binding sites in urine through oxidation, leading to false negative results in drug detection and immunological detection); or detect the pH value, specific gravity, temperature, etc., of urine (these indicators are mainly used to test whether it is diluted with liquids, such as water, artificial urine).

Thus, in traditional drug abuse testing, the analyte is tested by immunological methods, and the device is also equipped with chemical methods to test for urine adulteration, which brings many disadvantages.

First, chemical testing generally involves redox reactions, which produce new substances. When contacting urine, these substances may dissolve into the urine, which may interfere with the principle of antibody-antibody binding, thereby causing inaccurate testing of the analyte. However, the present invention uses immunological methods to test both the analyte and the adulteration marker, which do not affect each other.

Second, in actual production and assembly, immunological methods and chemical methods are two separate production lines that cannot be merged. Some reagents of chemical methods are volatile and will affect the performance of immunological reagents. However, the present invention uses immunological methods to test for urine adulteration, which can be merged with the immunological methods used to test for analytes in urine into one production line, reducing costs.

Third, if the chemical method for urine adulteration testing and the immunological method for analyte testing is set in one device, such as a test card that includes both the analyte for immunological testing and the reagent for chemical reaction testing for urine adulteration, it is necessary to set different spaces to accommodate the reagents for immunological testing and chemical testing. When both are lateral flow test strips, the immunological test and chemical test have different specifications, sizes, and different design requirements for space, which requires designing two different spaces, increasing the design difficulty and production cost. However, using immunological methods to test both urine adulteration and analytes can be uniformly designed to the same specifications and sizes, making the production cost simpler. In addition, placing the chemical method for urine adulteration testing and the immunological method for analyte testing in one space will affect the effectiveness of the respective reagents during subsequent storage and packaging, causing cross-contamination.

Fourth, commercial artificial urine has most of the properties of naturally excreted human urine, such as pH and specific gravity similar to those of naturally excreted human urine. When testing pH or specific gravity and other chemical methods cannot identify it is artificial urine or natural urine. But, some endogenous small molecular metabolites of the present invention can be easily tested, and immunological methods can successfully test and effectively identify them. These small molecular compounds cannot be tested by chemical reactions.

Even if some exogenous bleaching agents, oxidizing agents, and the like are added, the content of endogenous metabolites generally cannot be altered. In some embodiments, such agents may, at most, damage the binding sites of the endogenous metabolic adulteration markers with antibodies, similar to how they damage the binding sites of analytes. However, following such damage, an immunoassay will fail to detect the adulteration markers or yield a negative result, which is also indicative of adulteration of naturally produced urine, consistent with results obtained by chemical testing methods. In some preferred embodiments, however, the immunoassay of the present invention for detecting endogenous markers to identify urine adulteration by dilution or substitution with artificial urine can avoid false-negative results associated with conventional chemical methods for measuring pH and specific gravity.

Therefore, the present invention uses immunological methods to test urine adulteration markers (especially some small molecular endogenous compounds that cannot be tested by traditional chemical testing), and combines them with the same immunological methods to test exogenous analytes, which can effectively avoid many disadvantages of traditional chemical methods for urine adulteration testing and immunological testing for analytes.

### Test Element

As used herein, the term “test element” refers to any element capable of detecting whether a sample contains an analyte of interest, and also capable of testing adulteration markers to determine whether urine is adulterated. Such detection may be based on any technical principle, including immunology, chemistry, electronics, optics, molecular biology, nucleic acid technology, physics, etc. The test element may be a lateral flow test strip, which can detect multiple analytes. Of course, other suitable test elements may also be used in the present invention. In some preferred embodiments, lateral flow combined with immunological methods is used to test the urine adulteration markers and analytes in urine of the present invention.

Various test elements may be used in combination in the present invention. One form is a test strip. Test strips for analyzing analytes (such as drug abuse substances) or urine adulteration markers in urine samples may be in various forms, such as immunoassay formats. The test strip may adopt a non-competitive or competitive analysis mode. A test strip generally includes an absorbent material having a sample application zone, a reagent zone, and a test zone. After the sample is added to the sample application zone, it flows to the reagent zone through capillary action. In the reagent zone, if the analyte is present, the sample binds to the reagent. The sample then continues to flow to the test zone. Other reagents, such as molecules that specifically bind to the analyte, are immobilized in the test zone. These reagents react with the analyte in the sample (if present) and bind the analyte in this zone, or bind to a reagent in the reagent zone. Labels used to display detection signals are present in the reagent zone or a separate labeling zone.

A typical non-competitive analysis mode is: if the sample contains the analyte or its content is higher than a preset threshold, a signal is generated, indicating a positive result; if the sample does not contain the analyte or its content is lower than the preset threshold, no signal or a very weak signal is generated, indicating a negative result. In the competitive method, if the analyte is not present in the sample or its content is lower than the preset threshold, a signal is generated, indicating a negative result; if the analyte is present or its content is higher than the preset threshold, no signal is generated, indicating a positive result. For adulteration markers, if the urine contains the adulteration marker or the concentration of the adulteration marker is within the “normal concentration range” of the adulteration marker, it indicates that the urine is not adulterated, and the detection result is a positive result; if the urine does not contain the adulteration marker or the concentration of the adulteration marker is within the “adulteration concentration range” of the adulteration marker, it indicates that the urine is adulterated, and the detection result is a negative result.

For competitive testing using chromatographic principles, a positive result means that the test line does not appear or appears at a certain concentration; when the test line appears or appears above a certain concentration, it is a negative result.

The test element may be a test strip, which may be made of absorbent or non-absorbent materials. The test strip may include a variety of materials for liquid sample transfer. One material of the test strip may be covered on another material, such as filter paper covered on a nitrocellulose membrane. One zone of the test strip may be made of one or more materials, while another zone may be made of one or more other different materials. The test strip may be adhered to a support or a rigid surface to improve the strength of holding the test strip.

The analyte is detected by a signal generation system, such as using one or more enzymes that specifically react with the analyte. Using the method of immobilizing specific binding substances on the test strip as described above, a composition of one or more signal generation systems is immobilized in the analyte detection zone of the test strip. The substance that generates the signal may be in the sample application zone, the reagent zone, the test zone, or the entire test strip, and the substance may fill one or more materials of the test strip. A solution containing the signal substance is added to the surface of the test strip or one or more materials of the test strip are immersed in a solution containing the signal substance. The test strip added with the solution containing the signal substance is dried. Signal generation here includes colored particles, colored water-soluble dyes, or invisible light labeling substances (such as fluorescence).

The various zones of the test strip may be arranged in the following order: sample application zone, reagent zone, test zone, control zone, and liquid sample absorption zone. The control zone is located downstream of the test zone. All zones may be arranged on a single test strip using only one material; alternatively, different zones may use different materials. Each zone may be in direct contact with the liquid sample, or different zones may be arranged according to the flow direction of the liquid sample, with the end of each zone connected to and overlapping with the front end of another zone. The materials used may be materials with good water absorption, such as filter paper, glass fiber, or nitrocellulose membrane. The test strip may also adopt other forms.

Generally, commonly used test strips are nitrocellulose membrane test strips, that is, the test zone includes a nitrocellulose membrane on which specific binding molecules are immobilized to display the detection result; it may also be a cellulose acetate membrane or nylon membrane, etc. For example, the test strips or devices containing test strips described in the following patents: U.S. Pat. Nos. 4,857,453; 5,073,484; 5,119,831; 5,185,127; 5,275,785; 5,416,000; 5,504,013; 5,602,040; 5,622,871; 5,654,162; 5,656,503; 5,686,315; 5,766,961; 5,770,460; 5,916,815; 5,976,895; 6,248,598; 6,140,136; 6,187,269; 6,187,598; 6,228,660; 6,235,241; 6,306,642; 6,352,862; 6,372,515; 6,379,620; and 6,306,642. The test strips and similar devices with test strips disclosed in the above patent documents can all be used in the test element or detection device of the present invention to detect analytes, such as the detection of analytes in samples.

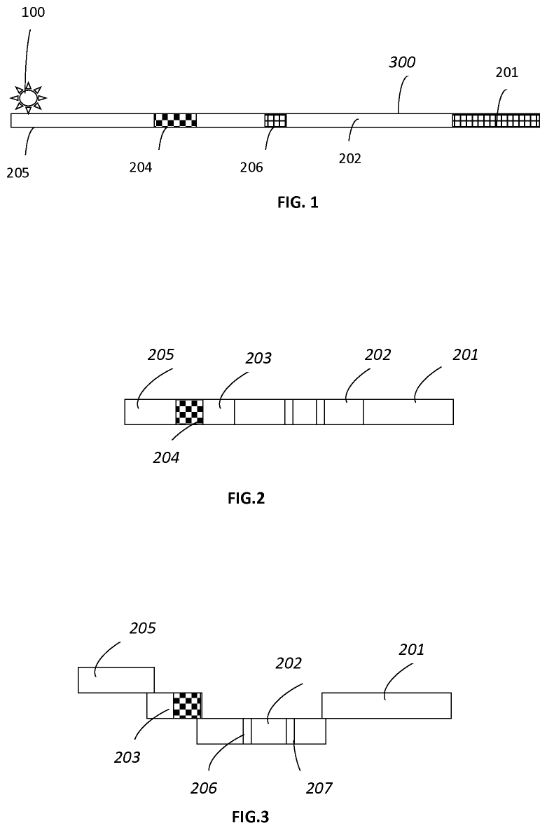

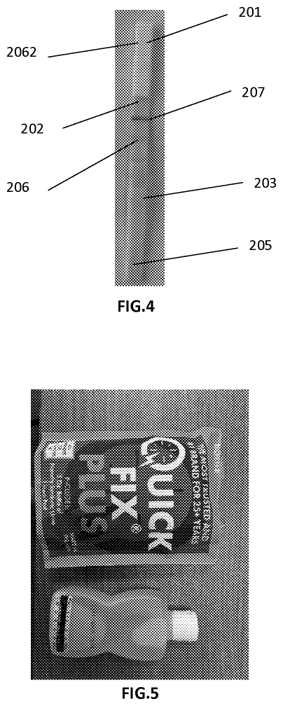

The detection test strips used in the present invention may be the commonly referred to lateral flow test strips. The specific structure and detection principle of these detection test strips are well-known technologies to those of ordinary skill in the art in the prior art. A common detection test strip includes a sample collection zone or sample application zone 205, a labeling zone 204, a detection zone 202, and an absorption zone 201. The sample collection zone includes a sample receiving pad, the labeling zone includes a labeling pad, and the absorption zone may include an absorbent pad. The detection zone includes necessary chemical substances capable of detecting the presence or quantity of analytes or adulteration markers, such as immunological reagents or enzymatic chemical reagents. Generally, commonly used detection test strips are nitrocellulose membrane test strips, that is, the detection zone includes a nitrocellulose membrane on which specific binding molecules are immobilized to display the detection result in the detection result zone 206; it may also be a cellulose acetate membrane or nylon membrane, etc. Of course, a detection result control zone 207 may also be included downstream of the detection result zone 206. Usually, the control zone and the detection zone appear in the form of horizontal lines, which are the test line (T line 206) or the control line (C line 207), Figure 1-3, Figure 4.6.7 of the test strip. Such detection test strips are traditional test strips, and of course, may also be other types of test strips that use capillary action for detection. In addition, the detection test strip generally contains dry chemical reagent components, such as immobilized antibodies or other reagents. When encountering liquid, the liquid flows along the test strip through capillary action. As it flows, the dry reagent components dissolve in the liquid, and then react with the dry reagents in the next zone to perform necessary detection. Liquid flow is mainly carried out through capillary action. All of these can be used in the detection device of the present invention, or placed in the detection chamber to contact the liquid sample, or used to detect the presence or quantity of analytes in the liquid sample entering the detection chamber. The test element is generally arranged in the test chamber. When the test chamber contains a fluid sample, the fluid sample contacts the test element and undergoes analysis or detection.

Using the above traditional methods for detecting analytes in urine samples to test adulteration markers in urine all adopt immunological methods, with only different interpretations of the results. A positive or negative result for the analyte indicates the presence or quantity of the analyte, while a positive or negative result for adulteration indicates that the urine is not adulterated or is adulterated.

Neopterin and analogous substance thereof.

In some embodiments, the inventors of the present invention have found that an abnormal elevation of neopterin (“NPT” for short) (chemical formula C9H11N5O4, CAS accession number 2009-64-5, molecular weight 253.2147) in the human body indicates that the individual has an overall health risk, or is overall unhealthy, and is at increased risk of developing a particular disease, particularly a tumor or cancer, or/and an infectious disease, if not intervened early. Thus, it is suggested that the subject needs to further detect specific markers associated with tumors or antibodies or antigens associated with infectious diseases, or other specific markers associated with tumor cancer or infectious diseases. As explained earlier, if the level of NPT is higher than a normal level, it does not mean that the individual has a malignant tumor or cancer, or/and an infectious disease, but merely that there is a risk of developing a malignant tumor or cancer, or/and an infectious disease without intervention. If the result is positive and timely intervention is carried out to make the overall unhealthy status of the individual become overall healthy and NPT become negative, the risk of developing a malignant tumor or cancer, or/and an infectious disease is greatly reduced. Generally, there is a certain concentration of NPT in the urine of the human body. This concentration is generally stable or constant. If NPT is abnormally increased, it means that the human body is overall unhealthy and has an increased risk of suffering from a certain disease. This disease may be cancer or a tumor, or an infectious disease, such as an acute infection.

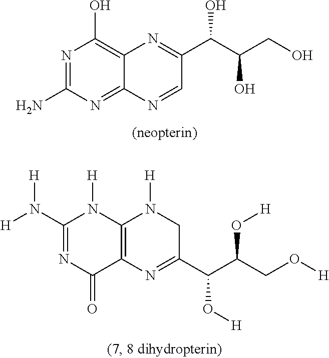 [SMILES: Nc1nc(O)c2nc([C@H](O)[C@H](O)CO)cnc2n1 ; Nc1nc(=O)c2c([nH]1)NCC([C@@H](O)[C@@H](O)CO)=N2]

Neopterin and 7, 8 dihydropterin are the predominant pterins found in urine, of which neopterin accounts for 25-45% of total pterins. Neopterin and 7, 8 dihydropterin are the predominant pterins found in urine, of which neopterin accounts for 25-45% of total pterins (Wächter et al., 1992, Lindsay et al., 2014). 7, 8 dihydropterin is the main component of the rest of pterins found in urine. Both are produced by macrophages in immune system activation and inflammatory responses and are used as clinical markers of monocyte/macrophage activation. However, the present invention can employ both substances as markers of overall health. During testing, antibodies can be selected to recognize the main common functional groups of the two substances. Of course, antibodies that bind or recognize neopterin but do not recognize 7, 8 dihydropterin can also be selected. Therefore, 7, 8 dihydropterin is also a marker that can be used as a marker of the present invention to predict whether or not an individual is healthy and the health status of an individual.

In healthy adults, the mean urinary concentration of neopterin is approximately 125 μmol/mol creatinine. The urine concentration in urine is usually measured by creatinine to compensate for dilution of urine. The upper limit of normal neopterin (μmol)/creatinine (mol) in females is: 208 (18-25 years), 209 (26-35 years), 239 (36-45 years), 229 (46-55 years), 249 (56-65 years), 251 (over 65 years). The normal upper limit in males is slightly lower: 195 (18-25 years), 182 (26-35 years), 176 (36-45 years), 197 (46-55 years), 218 (56-65 years), 229 (over 65 years). These upper limits include 97.5% of healthy controls. The creatinine concentration varies widely depending on the subject's degree of hydration, but ranges around 12 mmol/l. A peak occurs late at night and early in the morning. Thus, the neopterin concentration detected in the first morning urine was approximately 1500 nmol/L (379 ng/mL) (Wachter et al., 1989, Lindsay et al., 2014). The above concentration can be selected as a reference level or cut-off value for healthy people, and individuals above this cut-off value or reference level may have unhealthy performance. Of course, the higher the concentration or abnormally higher than the reference level, the overall health status is indicated to be poor or unhealthy, and in particular, treatment and intervention are needed, so as to reduce the risk of developing tumors or cancer, or/and an increased risk of infectious diseases.

### Neopterin and Adulteration

In some embodiments, NPT or 7,8-dihydropterin of the present invention can also be used as markers for natural urine adulteration. Traditional urine adulteration testing measures the specific gravity, pH value, etc., of urine to determine whether urine is adulterated. Urine adulteration is an abnormal behavior but often occurs in actual testing. For example, during recruitment, job applicants are often required to undergo drug abuse testing, but applicants do not want to test positive, so they adulterate the urine, such as diluting the urine by adding a large amount of water to the collected urine, reducing the concentration of analytes in the applicant's urine to undetectable levels. In addition, simply not collecting real urine but replacing it with other solutions, so-called artificial urine, will also lead to incorrect test results. In human urine, NPT or 7,8-dihydropterin maintains a normal concentration range. If during testing, NPT is below the normal concentration or cannot be detected, it indicates that the urine may be adulterated. Using this indicator to test urine adulteration is much simpler than the conventional traditional testing of specific gravity and pH, providing a new method for testing urine adulteration. Of course, if the detected NPT concentration is abnormally increased, it indicates that the urine is not adulterated, but also indicates that the individual may be overall unhealthy, with an increased risk of developing cancer and acute infectious diseases in the later stage. However, under the condition of excluding the possibility of artificially injecting or adding NPT to the urine, the suspicion or possibility of urine adulteration can be judged. This testing method is performed using immunological testing.

When the target of adulteration is artificial urine, the detection of NPT in the artificial urine indicates that the artificial urine has been adulterated. If the presence of NPT is not detected in the artificial urine, this indicates that the artificial urine has not been adulterated.

### Dopamine

Compared with physical diseases or health conditions, mental health has attracted increasing attention. The inventors have found that for some mental health conditions, the following 4 substances are commonly used markers of mental health, such as dopamine (abbreviated as “DOP”) (dopamine is a compound obtained by hydroxylation of phenethylamine, chemical name: 3,4-dihydroxyphenethylamine. Its molecular formula is C8H11NO2, and its relative molecular mass is 153.18 g/mol), serotonin; cortisol; epinephrine. The invention found that dopamine can also be used as a marker to test urine adulteration. For example, when the detected concentration of DOP in urine is lower than the normal range, it indicates that the urine is diluted and may be adulterated.

When the target of adulteration is artificial urine, the detection of DOP in the artificial urine indicates that the artificial urine has been adulterated. If the presence of DOP is not detected in the artificial urine, this indicates that the artificial urine has not been adulterated

### Setting of Threshold (Cut Off)

In some embodiments, the threshold for urine adulteration may vary among different countries, different living environments, and different age groups. However, the average threshold level of urine adulteration markers can be tested or screened clinically. If these adulteration markers are higher than these threshold levels, it indicates that the urine is not adulterated; when the tested value is lower than the threshold, it indicates that the urine is adulterated. Therefore, the threshold is generally set according to the “normal range” of the adulteration marker. For example, if the concentration range of a certain adulteration marker in urine is 100-250 mg/mL, the threshold can be set at 100 mg/mL. When it is less than the threshold, it indicates that the urine is adulterated; when it is greater than the threshold, it indicates that the urine is not adulterated. Such adulteration behavior includes at least urine dilution or replacement with commercial artificial urine.

However, if the target of adulteration is artificial urine, the “normal range” for the threshold shall be zero or less than a threshold value. A test result within the normal range indicates that the artificial urine is not adulterated, whereas a result above the threshold indicates that the artificial urine is adulterated.

Of course, in some embodiments, when the concentration of the adulteration marker exceeds the normal range or the set threshold, it indicates that the individual is unhealthy or has a certain disease. In some embodiments, the threshold of Dopamine in urine can be about 200 ng/mL, the threshold of HCY in urine can be 2000 ng/mL, the threshold of mALB in urine can be 40 μg/mL, and the threshold of DOP in urine can be 500 ng/mL. If the tested values are all lower than the above set thresholds, it may indicate urine adulteration behavior, especially that the urine has been diluted in advance; if higher than these thresholds, it indicates that the urine is not adulterated. It can be understood that these thresholds are only estimated values and may be adjusted according to clinical requirements.

### Antibodies

Antibodies used in the immunological testing of the present invention can be any antibodies that can specifically bind to analytes or adulteration markers in the sample, especially the 4 marker substances used in the present invention to test the overall health of an individual or as adulteration markers: neopterin (NPT); cystine, homocysteine (HCY); dopamine (DOP); or microalbumin. For example, NPT and DOP can be used both as adulteration markers and as markers of health status. For example, if the detected concentration is much lower than the normal concentration of NPT or DOP, it indicates that the urine is adulterated; when the detected concentration is higher than the normal concentration, it indicates that the human body is unhealthy and also indicates that the urine is not adulterated.

Antibodies that can be used as binding agents can be any antibodies known to those skilled in the art. “Antibody” refers to immunoglobulin molecules and antigen-binding portions of immunoglobulin molecules, i.e., molecules containing antigen-binding sites that specifically bind to analytes, analyte analogs, or ligands (“immune reaction”). The term also includes derivatives of antibodies that retain binding ability, and any proteins containing binding domains homologous or substantially homologous to the binding domains of immunoglobulins. These proteins may be derived from natural substances or may be partially or fully synthetic. An antibody may be monoclonal or polyclonal. An antibody may be a member of any immunoglobulin type, including any human immunoglobulin type: IgG, IgM, IgA, IgD, and IgE. “Antibody fragment” is a derivative of an antibody or a portion of an antibody that is less than full-length. An antibody fragment can retain at least one significant site of the binding ability of the full-length antibody. Examples of antibody fragments include Fab, Fab′, F(ab′)2, scFv, Fv, dsFv dimers, and Fd fragments, but are not limited to the above. Antibody fragments can be generated by any method. For example, antibody fragments can be generated by enzymatic or chemical cleavage of a complete antibody, or by recombinant genes encoding partial antibody sequences. In other words, antibody fragments can be produced partially or fully recombinantly. Antibody fragments can be any single-chain antibody fragments. In other words, antibody fragments can contain multiple interconnected peptide chains, for example, connected by disulfide bonds. Antibody fragments can also be any multimolecular complex. A functional antibody fragment typically contains at least about 50 amino acids, and more antibody fragments typically contain at least about 200 amino acids. Single-chain Fvs (scFvs) are recombinant antibody fragments consisting of only a variable light chain (VL) and a variable heavy chain (VH) covalently linked to each other by a polypeptide chain. One of VL and VH has an amino-terminal region. The length and composition of the polypeptide chain are variable, and its length can bridge the two variable domains without seriously affecting the arrangement of atoms. The polypeptide chain is usually mainly composed of extended glycine and serine residues, with some glutamic acid and lysine residues scattered to increase its solubility. A “dimer” refers to a dimer of single-chain Fvs. The monomers of the dimer usually contain shorter peptide chains than most single-chain Fvs, and they show a tendency to form dimers.

“Fv” fragments consist of one VH and one VL domain connected to each other non-covalently. The term “dsFv” as used herein refers to an Fv containing an intermolecular disulfide bond that stabilizes the VH-VL pair. “F(ab′)” fragments are fragments of antibodies that are essentially the same as those obtained by digesting immunoglobulins (usually IgG) with pepsin at pH 4.0-4.5. This fragment can also be produced recombinantly. “Fab′” fragments are antibody fragments that are essentially the same as those obtained by reducing the disulfide bonds connecting the two heavy chains on the F(ab′) fragment. Fab′ fragments can also be produced recombinantly. “Fab” fragments are antibody fragments that are essentially the same as those obtained by digesting immunoglobulins (usually IgG) with papain. Fab fragments can also be produced recombinantly. The heavy chain fragment on the Fab fragment is the Fd fragment.

In the present invention, the antibody can specifically bind to analytes or adulteration markers in the liquid sample, such as urine. The analytes here are exogenous substances, such as drug abuse, and the adulteration markers are endogenous substances. In some embodiments, the antibody is also labeled with a labeling substance, which is a substance that displays the quantity of the antibody and can be read by equipment or the naked eye. The labeling substance can be colored particles, such as gold particles, latex particles, fluorescent substances, etc. These colored particles can be directly observed and read by the naked eye, or can be read by photography. Other labeling substances, such as fluorescent substances or others, can be read by equipment.

In some embodiments, the antibodies of the present invention that specifically bind to analytes or markers among adulteration markers are labeled with labeling substances, which are processed and sprayed on the labeling pad and exist in a dry state. When the labeling pad is wetted by the liquid sample, it can dissolve in the liquid sample and flow downstream of the test strip along with the liquid sample. When the sample contains the above-mentioned analytes or adulteration markers, the antibody can bind to the marker substances to form a complex, for example, forming such complexes: labeling substance-anti-NPT antibody-NPT (in urine), labeling substance-anti-analyte antibody-analyte or metabolite (in urine). The antibodies here are monoclonal antibodies. These complexes flow to the test zone, and the analytes in the urine compete with the immobilized analytes or analogs of adulteration markers on the test zone to bind to the antibodies. Generally, the higher the concentration of the analyte in the sample, exceeding the preset threshold, the more the test zone does not show a line, indicating a positive result; when the concentration is lower than the preset threshold, a line appears, indicating a negative result.

### Analogs of the Analyte or Adulteration Marker Substance

The substances herein include the analyte and the adulteration marker, as well as their analogs. Such analogs can form complexes with antibodies, and the antibodies can also form complexes with the analyte. The analytes herein refer to the adulteration markers and abused drugs of the present invention. In some embodiments, the analog of the analyte exhibits an ability to competitively bind to the antibody on the test region with the analyte, or exhibits the same antibody-binding ability as the analyte, or the two have different antibody-binding abilities. In some embodiments, the analog of the adulteration marker substance exhibits an ability to competitively bind to the antibody on the test region with the adulteration marker substance, or exhibits the same antibody-binding ability as the adulteration marker substance, or the two have different antibody-binding abilities.

The binding ability between the analog of the adulteration marker substance and the antibody may be stronger or weaker than that between the adulteration marker substance and the antibody. The binding affinity between the analog of the adulteration marker substance and the antibody may be higher or lower than that between the adulteration marker substance and the antibody. Such binding ability may be selected or optimized according to test needs using known techniques in the art. For example, binding molecules, such as binding sites or members of a binding pair, may be obtained by random or non-random mutagenesis.

The analog of the adulteration marker substance may be a fragment of the marker that retains certain sites of the analyte. A labeling substance may be attached to such analogs via a linker, wherein the linker comprises a binding site that is not present on the adulteration marker substance, the analog of the adulteration marker substance, or the labeling substance. For example, such sites may be recognized by certain antibodies but not by the adulteration marker substance, its analog, or the labeling substance.

Alternatively, such linkers may be recognized by antibodies without being recognized by sites on other linkers. Such linkers include, but are not limited to, proteins, natural or synthetic polypeptides, or carbohydrates, such as keyhole limpet hemocyanin (KLH), bovine gamma globulin (BGG), bovine serum albumin (BSA), bovine thyroglobulin (BTG), ovalbumin (OVA), sperm whale myoglobin (SWM), tetanus toxoid (TT), methylated bovine serum albumin (mBSA), and rabbit serum albumin (RSA).

In some aspects of the present invention, the analog of the adulteration marker substance may be an antigen, while the adulteration marker substance itself is a hapten. To render the marker capable of eliciting an immune response, the marker needs to be modified to become an antigen by covalently attaching certain molecules to confer immunogenicity. Such molecules may be any of the proteins in the aforementioned linkers.

In some aspects, the analogs of the markers of the present invention are immobilized on the test region and cannot move with the flow of liquid. These analogs competitively bind to the antibody conjugated with a labeling substance with the analyte. Accordingly, on the test region, absence of a colored line indicates a positive test result, and presence of a colored line indicates a negative test result. That is, a positive result indicates that the marker substance in the sample exceeds the predetermined threshold, and a negative result indicates that the marker substance in the sample is below the predetermined threshold.

### Flow of Liquid

Flow of liquid generally refers to the movement of liquid from one location to another. Under normal circumstances, most liquid flow in nature occurs from a higher position to a lower position driven by gravity. The flow described herein also relies on an external force, namely external gravity, and may be referred to as natural gravity flow. In addition to gravity, liquid can also flow from a lower position to a higher position against gravity. For example, liquid may move from a lower position to a higher position by means of liquid extraction, liquid compression, or application of pressure on the liquid; alternatively, such flow may occur by overcoming the liquid's own gravity due to pressure.

### Example 1 Preparation of the Immunological Lateral Flow Detection Device Provided

The immunological lateral flow detection device prepared in this example is shown in FIGS. 3, 4, 6, and 7, including a test strip. According to the liquid flow direction, from upstream to downstream, it is sequentially a sample zone 205, a labeling zone 203, a detection zone 202, and an absorption zone 2014; wherein the absorption zone is prepared using general absorbent filter paper as the absorbent pad; the sample zone 203 uses a sample application pad made of glass fiber, so that the liquid sample is applied to the glass fiber, and the liquid sample flows to the labeling pad on zone 203 through capillary action; the labeling zone is made into a labeling pad, including antibodies conjugated with labeling particles (such as gold particles, fluorescent particles, latex particles, dyes, or other colored labeling substances), and then the labeling mixture is sprayed on a polyester membrane using spraying equipment to make the labeling pad. The labeling substances on the labeling pad can flow with the liquid flow; the detection zone uses a nitrocellulose membrane. The analog of the adulteration marker for the test line is dissolved in PBS buffer solution, and then the nitrocellulose membrane is streaked using membrane spotting equipment, and then the nitrocellulose membrane is placed in an oven to dry for later use. The adulteration marker or its analog treated on the membrane is generally immobile. The detection zone includes a detection line (test line 206) and a detection result control zone (control line 207).

After preparing the absorption zone 201, sample zone 205, labeling zone 203, and detection zone 202 respectively, assembly is performed: one end of the sample application pad is overlapped on one end of the labeling pad, the other end of the labeling pad is overlapped on the nitrocellulose membrane, and the nitrocellulose membrane at one end of the control line is overlapped with absorbent filter paper, thus forming the entire detection test strip.

When the sandwich method is used to make the test element, it can be a direct double-antibody sandwich method or an indirect method. For example, to detect the adulteration marker antigen A1 in the urine sample, the first antibody AB1 against antigen A1 conjugated with a color label can be treated on the labeling pad, and the second antibody AB2 against antigen A1 can be immobilized on the test line. The more antigen A1 in the sample, the more labeled substances captured on the test line, the darker the color, indicating a higher content and a positive result. When the concentration of the adulteration marker in the urine is very low, lower than the normal range or absent, a negative result appears, indicating that the urine may be adulterated.

In other embodiments, when the competitive method is used for testing, for example, it is desired to detect adulteration markers in urine. For example, NPT is used as an adulteration marker for testing. Monoclonal antibodies that specifically bind to NPT in urine sample are treated on the labeling zone, and gold particles are conjugated to the antibodies. NPT or an analog of NPT (linked to NPT-BAS) is immobilized on the test line. During testing, if NPT is present in the urine or higher than the preset threshold, the antibody first forms a complex with NPT in the urine: labeling substance-antibody-NPT, which then flows to the detection line in the test zone. The NPT in the urine competes with the NPT or its analog immobilized on the test line to bind to the antibody. The more NPT in the urine, the more antibodies are occupied by NPT in the urine, and the fewer antibodies are captured by NPT on the test line, thus the lighter the color of the line, indicating a positive result. On the contrary, if there is no NPT or the concentration is very low in the urine, more antibodies are captured on the test line, and the color of the line is darker, indicating a negative result.

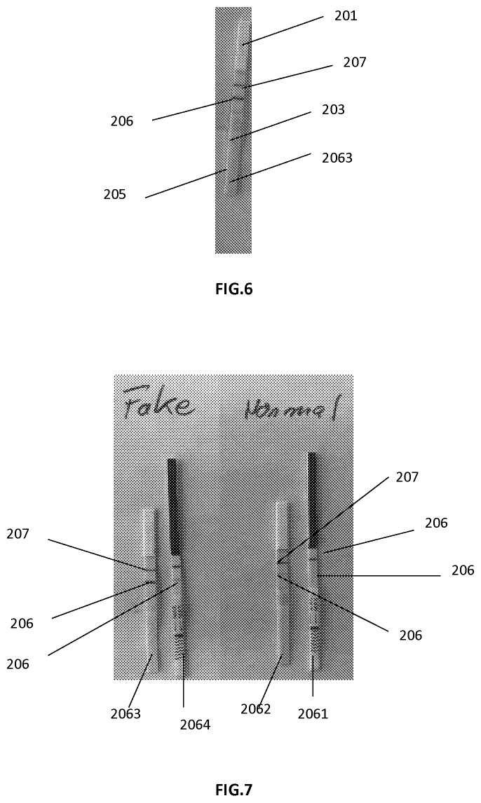

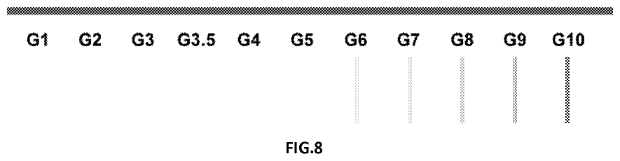

As shown in FIG. 4, when testing urine naturally excreted by normal humans using the competitive method, the color depth of the T line 206 is approximately G3, indicating a positive result and that the urine is not adulterated. When the same test strip is used to test commercial artificial urine, the color of the T line is G8, showing a negative result, indicating that the adulteration markers as present invention is not existed in the artificial urine, that can be looks as a adulteration result (as shown in FIG. 6). However, when testing the pH and specific gravity of artificial urine using traditional chemical methods, the result is positive, indicating that the artificial urine is urine naturally excreted by humans and is not adulterated. As shown in FIG. 8, which is a color chart for gold labeling, generally, when the color of the T line is below G3, it is a positive result; when it is above G3, it is a negative result (competitive method). Of course, if the non-competitive method (sandwich method) is used, the color below G3 is a negative result, and the color above G3 is a positive result.

### Example 2 Using Neopterin (NPT) as an Adulteration Marker to Test Whether Natural Urine is Adulterated

Similarly, 100 mL of naturally excreted human urine was mixed with 100 mL of artificial urine to form 200 mL of mixed urine 1; similarly, 100 mL of naturally excreted urine was added with 100 mL of water to form 200 mL of diluted urine 2; then 200 mL of naturally excreted human urine 3 was provided. The test strips of Example 1 were used to test the three types of urine. It was found that the T line color of mixed urine 1 was G8 (FIG. 6), and the T line color of diluted urine 2 was G10, indicating that both mixed urine 1 and 2 were negative results, indicating that the urine was adulterated. The T line of urine 3 was G3 (FIG. 4), indicating a positive result. This indicates that the concentration of NPT was diluted, showing a negative result, indicating that the urine was adulterated. The undiluted and unadulterated urine showed a positive result, indicating no adulteration.

### Example 3 Using DOP as an Adulteration Marker to Test Whether Urine is Adulterated (Competitive Method)

Similarly, 100 mL of naturally excreted human urine was mixed with 100 mL of artificial urine to form 200 mL of mixed urine 1; similarly, 100 mL of naturally excreted urine was added with 100 mL of water to form 200 mL of diluted urine 2; then 200 mL of naturally excreted human urine 3 was provided.

At the same time, DOP in the urine samples was tested with reference to the method of the example. When the competitive method was used for testing, for example, when it was desired to detect abused drugs in urine or saliva, such as DOP (a small drug molecule, hapten—almost no immunogenicity), antibodies corresponding to the DOP antigen conjugated with color particles were treated on the labeling pad, and DOP antigen or its analog was treated on the detection line. When the sample contains DOP, DOP binds to the antibody of the color particles. When flowing to the detection line, the antigen on the detection line competes with the analyte in the sample to bind to the antibody. Thus, the higher the content of small molecules in the sample, the fewer color particles captured on the detection line, and the lighter the color, indicating a positive result; on the contrary, the lower the content of small molecules in the sample, the more color particles captured on the detection line, and the darker the color, indicating a negative result. The naturally excreted human urine contained the drug abuse small molecule substance (DOP), and the test result was positive.

The difference between the adulteration test strip and Example 1 is that the test line immobilized DOP antigen, and the labeling zone was an antibody that specifically binds to DOP. Using such test strips to test the three types of urine, it was found that the T line color of mixed urine 1 was G7 (test strip 2064 in FIG. 7), and the T line color of diluted urine 2 was G9, indicating that both mixed urine 1 and 2 were negative results, indicating that the urine was adulterated. The T line of urine 3 was G2 (almost no line, as shown in test strip 2061 in FIG. 7), indicating a positive result. This also indicates that the concentration of DOP was diluted and reduced, showing a negative result, indicating that the urine was adulterated. The undiluted and unadulterated urine showed a positive result, indicating no adulteration. When the traditional analyte for testing THC was used to test the THC content, it was found that the test results of mixed urine 1 and 2 were negative (the T line colors were G7 and G8 respectively compared with the color chart), and urine 3 was positive (G2). This indicates that due to the adulteration of urine 1 and 2, THC could not be normally detected, indicating that the test result was not a true test result.

### Example 4 Using the Sandwich Method to Test Whether Urine is Adulterated

Urine peptides (molecular weight usually 2-10 kDa, a few up to 20 kDa) are mainly derived from plasma protein filtration, kidney/tissue protein degradation, and renal tubular secretion. In healthy humans, they are mainly collagen fragments, uromodulin fragments, albumin fragments, β2-microglobulin fragments, and small molecule functional peptides.

In this example, a uromodulin (T-H protein) fragment was selected: secreted by renal tubular epithelial cells, the most important specific peptide in the urine of healthy humans, with most fragments ranging from 2-8 kDa and a total content of 5-30 μg/mL.

The first antibody against the uromodulin fragment was treated on the labeling pad, the second antibody binding to the uromodulin fragment was immobilized on the test line, and the first antibody was conjugated with gold particles. The threshold was set at 5 μg/mL. Through the pairing of the first antibody and the immobilized antibody, values greater than the threshold were positive results, and values less than the threshold were negative results.

Similarly, 100 mL of naturally excreted human urine was mixed with 100 mL of artificial urine to form 200 mL of mixed urine 1; similarly, 100 mL of naturally excreted urine was added with 100 mL of water to form 200 mL of diluted urine 2; then 200 mL of naturally excreted human urine 3 was provided. The DOP test was positive.

The test results of urine 1-3 were tested with the adulteration test strip. It was found that the test results of urine 1-2 showed no lines, which were negative results. The T line colors were G2 and G3 respectively compared with the color chart FIG. 8, indicating that the urine samples were adulterated. A positive result was obtained in urine 3, and the T line color was G7 compared with the color chart FIG. 8. When the traditional analyte for testing DOP was used to test the DOP content, it was found that the test results of mixed urine 1 and 2 were negative (the T line colors were G7 and G8 respectively compared with the color chart), and urine 3 was positive (G2). This indicates that due to the adulteration of urine 1 and 2, DOP could not be normally detected, indicating that the test result was not a true test result.

### Example 5: Testing for Adulteration of Artificial Urine Using Competitive Assay

For example, when testing conventional artificial urine as shown in FIG. 5 with immunochromatographic test strips based on the competitive assay for NPT or DOP, the results are both negative, and the color and color ratio of the T line are higher than G4. It indicates that there is no NPT or DOP in the artificial urine.

When NPT or DOP are added into the artificial urine to a concentration greater than 50 ng/mL for DOP, 0.02 ug/mL for NPT, and the immunological test strips for NPT or DOP are used with a test threshold of 20 ng/mL for DOP and 0.01 ug/mL for NPT the results are both positive, indicating that the artificial urine is adulterated and contains endogenous adulteration markers.

It will be appreciated that all of the urine adulteration markers of the present invention can be artificially added to synthetic urine (which conventionally does not contain the adulteration markers of the present invention) to mimic naturally excreted urine, thereby achieving urine adulteration. In particular, certain small-molecule compounds are deliberately added to synthetic urine at concentrations consistent with those found in urine excreted by natural individuals, thus a Homovanillic Acid as the test subjects in adulterating urine samples. The term “synthetic urine” as used herein does not refer to urine excreted by natural individuals, but rather urine prepared by artificial means. For instance, lemon yellow pigment may be used to impart a urine-like color, and pH adjusters may be employed to adjust the pH within the range of naturally excreted human urine, such that it can be used to simulate naturally voided urine.

All patents and publications mentioned in the specification of the present invention indicate that these are disclosed techniques in the art and can be used by the present invention. All patents and publications cited herein are likewise listed in the references as if each publication is specifically and separately referenced. The present invention described herein may be implemented in the absence of any one or more elements, and one or more limitations, which are not specifically stated herein. For example, the terms “comprising”, “consisting essentially of” and “consisting of” in each of the examples herein may be replaced by the remaining two terms of one of the two. The term “one” herein only means “a”, and does not exclude the inclusion of only one, and may mean the inclusion of two or more. The terms and expressions employed herein are descriptive and are not limited thereto, and there is no intention herein to indicate that the terms and interpretations described herein exclude any equivalent features, but it can be noted that any appropriate changes or modifications can be made within the scope of the present invention and claims. It can be understood that the embodiments described in the present invention are preferred embodiments and features, and any person skilled in the art can make some modifications and changes based on the essence of the description of the present invention. These modifications and changes are also considered to be within the scope of the present invention and the scope limited by the independent claims and the dependent claims.

1. A device for detecting whether urine has been adulterated, said device comprising means for detecting the presence or quantity of an adulteration marker in the urine, wherein said device tests for the presence or quantity of the adulteration marker based on an immune reaction.

2. The device according to claim 1, wherein said immune reaction is conducted in a competitive assay format.

3. The device according to claim 1, wherein said immune reaction is conducted in a non-competitive assay format, which comprises a double-antibody sandwich assay format.

4. The device according to claim 2, wherein the test device comprises: a first antibody that specifically binds to the adulteration marker in the urine to form a complex, said first antibody being flowable with a liquid; and an adulteration marker or an analog thereof that is immobilized and non-flowable with the liquid; wherein adulteration marker in the urine competes with the immobilized adulteration marker or analog thereof for binding to the first antibody of the complex.

5. The device according to claim 3, wherein the adulteration marker is an endogenous metabolite present in urine naturally excreted by the individual.

6. The device according to claim 5, wherein the endogenous metabolite comprises a small-molecule compound selected from one or more as below: purine metabolites, pteridines and hormone or signaling molecule metabolites.

7. The device according to claim 5, wherein the endogenous metabolite comprises urea, a creatinine metabolite, creatinine, a pteridin metabolite, albumin, and urease.

8. The device according to claim 6, wherein the purine metabolite is selected one or more as below: adenine, guanine, hypoxanthine, or xanthine.

9. The device according to claim 7, wherein the pteridin metabolite is selected one or more as below: neopterin, biopterin, dihydroneopterin, dihydrobiopterin, sepiapterin, and 7-hydroxypterin.

10. The device according to claim 6, wherein the hormone or signaling molecule metabolite comprises dopamine or specific metabolites like homovanillic acid.

11. The device according to claim 4, wherein said device comprises a lateral flow test strip, and said immune reaction is performed on the lateral flow test strip.

12. The device according to claim 11, wherein the lateral flow test strip comprises a test region, and the adulteration marker or an analog thereof is immobilized in the test region.

13. The device according to claim 12, wherein the lateral flow test strip further comprises a labeled region that is flowable with the liquid, said first antibody is disposed in the labeled region, the labeled region is positioned upstream of the test region, and said first antibody is conjugated to a labeling substance.

14. The device according to claim 13, wherein the labeling substance comprises a color-forming substance and a fluorescent labeling substance, and the color-forming substance is selected one or more as below: metal particles and water-soluble dyes.

15. The device according to claim 14, wherein the adulteration marker has a normal concentration range in the urine.

16. The device according to claim 15, wherein when the urine is a urine that is naturally excreted from an individual, and wherein a result of detection of the adulteration marker being within the normal concentration range via the immune reaction indicates that the urine is unadulterated; and a result of detection of the adulteration marker being below the normal concentration range or within an adulterated range indicates that the urine has been adulterated.

17. The device according to claim 12, wherein when the urine is an artificial urine, and wherein a result of detection of the adulteration marker by the test strip is negative, that indicates the artificial urine is not adulterated; a result of detection of the adulteration marker by the test strip is positive, that indicates the artificial urine is adulterated.

18. The device according to claim 17, wherein the test region of the lateral flow test strip comprises a nitrocellulose membrane, and the adulteration marker or an analog thereof is immobilized on the nitrocellulose membrane.

19. The device according to claim 18, wherein the nitrocellulose membrane further comprises a control region downstream of the test region for validating a test result.

20. The device according to claim 19, wherein when the lateral flow test strip is used to test for adulteration of the urine, a negative result in the test region indicates that the urine is adulterated, and a positive result in the test region indicates that the urine is unadulterated.

21. The device according to claim 20, wherein the device further comprises a second lateral flow test strip for testing an analyte in the urine via a competitive assay, said analyte being a metabolite of an exogenous substance ingested by the individual and present in the urine.

22. The device according to claim 21, wherein the analyte is a drug of abuse.

23. The device of claim 1, wherein adulteration of the urine comprises dilution of urine naturally discharged or excreted by the individual, or substitution of urine naturally excreted by the individual with artificial urine.

24. A method for testing whether urine has been adulterated, said method comprising: testing an adulteration marker in the urine via an immunoassay; wherein the urine is unadulterated if the adulteration marker is within a normal range, and the urine is adulterated if the adulteration marker is within an adulterated range.

25. The method according to claim 24, wherein the adulteration marker is an endogenously metabolized small-molecule compound.

26. The method according to claim 25, wherein the small-molecule compound is selected from the group: pterin metabolites, signaling molecule metabolites, and hormone metabolites.

27. The method according to claim 26, wherein the method further comprises: providing a lateral flow test strip, said lateral flow test strip comprising a labeled region and a test region downstream of the labeled region; wherein the labeled region comprises a first antibody capable of binding to the adulteration marker in the urine to form a complex that is flowable with the urine, and the test region has the adulteration marker or an analog thereof immobilized thereon.

28. The method according to claim 27, further comprising: allowing the first antibody to specifically bind to the adulteration marker in the urine to form the complex; and allowing the adulteration marker in the urine to compete with the adulteration marker or analog thereof immobilized on the test region for binding to the first antibody.

29. The method according to claim 28, wherein an appearance of a colored line in the test region or a colored line is less than G3 when compared to a color chart, which indicates that the urine has been adulterated; and absence of color in the test region or a colored line is a reading less than G3 when compared to the color chart indicates that the urine is unadulterated.

30. The method according to claim 25, wherein the urine comprising a urine naturally excreted from an individual or an artificial urine.

31. The method according to claim 26, wherein the pterin metabolite comprises neopterin (NPT); dopamine resp or homovanillic acid.
